SEQUENCE LISTING (1 sequence)

SEQ ID NO: 1 - protein, 159 aa, synthetic construct
FQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADH

(19) United States
(12) Patent Application Publication
(10) Pub. No.: US 2026/0062689 A1
(43) Pub. Date: Mar. 5, 2026

(54) USE OF MULTITARGETED POLYPEPTIDE IN TREATMENT OF ORGAN FIBROSIS

(71) Applicant: NANFANG HOSPITAL OF SOUTHERN MEDICAL UNIVERSITY, Guangzhou City (CN)

(72) Inventors: YOUHUA LIU, Guangzhou City (CN); HANYING LUO, Guangzhou City (CN)

(21) Appl. No.: 19/028,816

(22) Filed: Jan. 17, 2025

(30) Foreign Application Priority Data
Aug. 30, 2024 (CN) 2024112053997

(51) Int. Cl. C12N 9/24; A61K 9/00; A61K 38/00; A61P 1/16; A61P 11/00; A61P 13/12; C12N 15/86

(52) U.S. Cl. CPC C12N 9/2402; A61K 9/0019; A61P 1/16; A61P 11/00; A61P 13/12; C12N 15/86; A61K 38/00; C12N 2750/14143; C12Y 302/01031

(57) ABSTRACT

The present disclosure relates to use of a multitargeted polypeptide in the treatment of organ fibrosis, in particular to use of a polypeptide mini-klotho and its gene therapy vectors and methods for the treatment of renal fibrosis, hepatic fibrosis, pulmonary fibrosis and other organ fibrosis. The present disclosure provides the use of mini-klotho in the treatment of renal, hepatic and pulmonary fibrosis models, with significant therapeutic effects and without obvious toxic or side effects. Therefore, the mini-klotho of the present disclosure can be prepared into a pharmaceutical preparation for the treatment of organ fibrosis.

### CROSS-REFERENCE TO RELATED APPLICATION

This application claims priority to Chinese Patent Application No. 202411205399.7, filed Aug. 30, 2024, the disclosure of which is hereby incorporated by reference in its entirety.

### INCORPORATION BY REFERENCE

The instant application contains a Sequence Listing encoded in xml format which was filed electronically by EFS-web and is hereby incorporated by reference in its entirety. Said xml format Sequence Listing created on Jan. 8, 2025 named “P24GZ1NW00152US-Sequence Listing.xml” and 2,003 bytes in size.

### TECHNICAL FIELD

The present disclosure relates to use of a multitargeted polypeptide in the treatment of organ fibrosis, in particular, to use of mini-klotho polypeptide and its gene therapy vectors in the treatment of renal fibrosis, hepatic fibrosis, pulmonary fibrosis and other organ fibrosis.

### BACKGROUND

Organ fibrosis is characterized by excessive deposition of extracellular matrix (ECM) due to dysregulation of tissue damage and repair response (Signal Transduct Target Ther. 2022; 7(1): 206), and primarily driven by myofibroblasts. Fibrosis occurs in many important organs, including kidney, liver, lung, heart, etc. Fibrosis-related diseases such as chronic kidney disease, liver cirrhosis, idiopathic pulmonary fibrosis, etc. have caused a huge disease burden worldwide. It has been reported that deaths caused by fibrosis-related diseases have reached 45% of all human deaths in developed countries (Nature 2020; 587 (7835): 555-566). However, there has been no effective treatments for fibrosis-related diseases so far. Therefore, organ fibrosis is increasingly recognized as a major human health challenge.

The pathological mechanisms of organ fibrosis are complex and have not yet been fully elucidated. However, TGF-β, FGF2, and Wnt/β-catenin pathways are recognized as the three key and important pathways that mediate the occurrence and development of fibrosis. TGF-β1 is one of the most potent fibrogenic factors and regulates fibrosis mainly through canonical and non-canonical signaling pathways. TGF-β1 can promote the activation of fibroblasts to myofibroblasts and the expression of α-smooth muscle actin (α-SMA), and induce apoptosis and epithelial-mesenchymal transition, resulting in ECM deposition. Wnt/β-catenin signaling can also promote organ fibrosis, in which Wnt ligands induce activation and overexpression of ECM in fibroblasts. FGF2 is also a key fibroblast factor that mainly promotes fibroblast proliferation and activation and induces epithelial-mesenchymal transition. In addition, it has been documented that TGF-β interacts with the Wnt/β-catenin pathway, and TGF-β can also induce FGF2 expression and activate the FGF2 pathway at the same time. It can be seen that TGF-β, FGF2, and Wnt/β-catenin pathways may promote each other. Therefore, a method that can simultaneously block the three signaling pathways of TGF-β, FGF2, and Wnt/β-catenin is of great significance and clinical application value in inhibiting the occurrence and development of organ fibrosis.

The present disclosure involves the discovery of a unique multitargeted polypeptide named mini-klotho. There are reports in the literature that klotho has the ability to bind TGF-β receptors, FGF receptors, and Wnt ligands and block TGF-β/Smad signaling (J. Biol. Chem., 2011, 10:8655-8665), Wnt/β-catenin signaling (J. Am Soc. Nephrol. 2013, 24:771-785), and FGF2/FGFR1 (J. Pathol. 2014, 234:560-572) signaling, thereby inhibiting organ fibrosis and delaying the progression of fibrosis-related diseases. However, a full-length anti-aging protein klotho (i.e., a transmembrane protein with a large molecular weight) is difficult and expensive to synthesize, and can cause side effects such as hypocalcemia and hypophosphatemia, making it difficult to apply in clinical practice.

The present disclosure has discovered that mini-klotho, as a novel and effective small molecule polypeptide, can alleviate the damage and fibrosis of important organs (such as kidney, liver, lung) and delay or/and reverse the course of fibrosis-related organ diseases.

The present disclosure further relates to the discovery of a unique mini-klotho gene therapy vector. Adeno-associated virus (AAV) is one of the most actively researched gene therapy vectors. As a vector, it has the advantages of high safety, low immunogenicity, wide host range, stable expression and physical properties. However, the commonly used gene therapy vectors AAV2 or AAV9 are mainly sequestered in the liver after intravenous injection, which may cause liver toxicity. In addition, high doses of AAV can also cause complement activation. The novel serotype Myo4A-AAV with myotropism has more efficient and selective muscle transduction with significantly reduced hepatic sequestration. As a weak division site, the muscle can become a long-term stable secretion site after being injected with the virus. Therefore, intramuscular injection treatment using Myo4A-AAV as a vector is a unique mini-klotho administration method due to its simple injection method, low dosage and other advantages.

Unilateral ureteral ligation (UUO) is a classic animal model of CKD, which can quickly and completely demonstrate different pathological stages and characteristics of interstitial fibrosis in obstructed kidneys, including inflammatory cell infiltration, proliferation and apoptosis of tubular cells, myofibroblast activation, and deposition of fibronectin and collagen I. The ischemia-reperfusion (IRI) model is a classic model of progression from acute kidney injury (AKI) to chronic kidney disease (CKD). Unilateral kidney ischemia-reperfusion injury (IRI) surgery at 10 days combined with contralateral nephrectomy (UIRI) can clearly demonstrate the pathological process of chronic renal fibrosis. The modeling method is simple and feasible, with a high success rate, and has the advantages of small surgical incision, short operation time and few complications. The established model is suitable for studying the progression of acute kidney injury to chronic renal fibrosis.

Carbon tetrachloride (CCL4) is one of the main drugs to induce hepatic cirrhosis.

The main characteristics of CCL4 model are oxidative stress, severe hepatic perilobular inflammation and septal fibrosis, as well as zone III and centrilobular necrosis. The modeling method is simple and feasible, with a high success rate, and is similar to the pathophysiology of human liver cirrhosis. The bleomycin (BLM) model is the most widely used model for inducing idiopathic pulmonary fibrosis (IPF). This model exhibits histological features of pulmonary fibrosis, such as loss of endobronchial granulation, collagen walls, and alveolar spaces, similar to those in patients with IPF. In addition, this model also has the advantages of simple and feasible modeling methods and high reproducibility, etc.

Pathologically, Masson's trichrome staining is usually used to observe the deposition of extracellular collagen in tissues. More specific tissue fibrosis is often observed and evaluated qualitatively and quantitatively by immunostaining and Western blot of makers of myofibroblasts such as α-smooth muscle actin (α-SMA), fibronectin and collagen I.

### SUMMARY

The object of the present disclosure is to provide use of a polypeptide (mini-klotho) in preparation of a drug for the treatment of organ fibrosis-related diseases. According to further features of the use of the present disclosure, the prepared drug includes: a therapeutically effective polypeptide (mini-klotho), and intramuscular injection treatments using the myotropic Myo4A-AAV novel adeno-associated virus (AAV) serotype as a vector.

The mini-klotho of the present disclosure can simultaneously inhibit three key signaling pathways of organ fibrosis: TGF-β, FGF2, Wnt/β-catenin. There have been no previous reports on mini-klotho polypeptide. The amino acid sequence structure of mini-klotho polypeptide is homologous to a sequence of an anti-aging protein (klotho) molecule. The present disclosure further aims to provide use of derivatives of polypeptide mini-klotho in the treatment of organ fibrosis-related diseases.

According to further features of the use described in the present disclosure, derivatives of mini-klotho include: shorter peptides containing the amino acid sequence of mini-klotho, mini-klotho related polypeptides with amino acid substitutions, and chemically modified mini-klotho and its shorter peptides.

According to the use, the prepared drug vector and method described in the present disclosure, Myo4A-AAV novel adeno-associated virus (AAV) serotype is used as a vector for intramuscular injection.

The experiments of the present disclosure prove that the mini-klotho polypeptide (mini-klotho) has no obvious toxic or side effects in mouse animal experiment. The inventor used the renal fibrosis model of unilateral ureteral obstruction (UUO) and unilateral ischemia-reperfusion injury (UIRI), the carbon tetrachloride (CCL4)-induced hepatic fibrosis model, and the bleomycin (BLM)-induced pulmonary fibrosis model to study the efficacy of mini-klotho in preventing and treating fibrosis in various organs. The results show that: in the renal fibrosis model, compared with the UUO or UIRI model group, the renal interstitial collagen deposition in the mini-klotho treatment group was significantly reduced, and a-smooth muscle actin and fibronectin were significantly reduced, indicating that mini-klotho can effectively inhibit renal interstitial fibrosis caused by UUO and UIRI. In the hepatic and pulmonary fibrosis models, compared with the CCL4 and BLM model groups, the collagen deposition and fibronectin in the liver and lung tissues of the mini-klotho treatment group were significantly reduced, indicating that mini-klotho had therapeutic effects on hepatic and pulmonary fibrosis.

In summary, mini-klotho can significantly inhibit fibrosis in various important organs (such as kidney, liver, lung) without obvious toxic and side effects, and thus can be used to prepare drugs that effectively inhibit organ fibrosis.

### BRIEF DESCRIPTION OF THE DRAWINGS

FIG. 1A, FIG. 1B and FIG. 1C show that mini-klotho inhibits the activation of rat renal fibroblasts (NRK-49F) in vitro. Rat fibroblasts (NRK-49F) are transfected with mini-klotho lentivirus to establish a stable cell line. They are treated with TGF-β, FGF2, and Wnt3a respectively. FIG. 1A shows that mini-klotho has an antagonistic effect on TGF-β-induced pathway downstream signals and the mediated fibrosis-related proteins. FIG. 1B shows that mini-klotho has an antagonistic effect on FGF2-induced pathway downstream signals and the mediated fibrosis-related proteins. FIG. 1C shows that mini-klotho has an antagonistic effect on Wnt3a-induced pathway downstream signals and the mediated fibrosis-related proteins.

FIG. 2 shows a structural diagram of mini-klotho adeno-associated virus overexpressing mini-klotho using Myo4A-AAV as a vector.

FIG. 3 shows a fluorescence staining image of green fluorescent protein (GFP) in various organs of mice. In the UUO mouse model, the mini-klotho AAV virus overexpressing green fluorescent protein (GFP) is injected in situ into the right quadriceps muscle, and the fluorescence intensity of the left and right muscles, left and right kidneys, heart, liver, spleen, lungs and other organs are detected.

FIG. 4 shows Masson's trichrome staining images of kidneys of mice in each group of the UUO model. In FIG. 4, from left to right are the sham operation group, sham operation+mini-klotho group, UUO group, and UUO+mini-klotho group.

FIG. 5 shows immunostaining images of renal fibrosis indicators (fibronectin and α-SMA) in mice in each group of the UUO model. In FIG. 5, from left to right are the sham operation group, sham operation+mini-klotho group, UUO group, and UUO+mini-klotho group.

FIG. 6 shows Masson's trichrome staining images of kidneys of mice in each group of the UIRI model. In FIG. 6, from left to right are the sham operation group, sham operation+mini-klotho group, UIRI group, and UIRI+mini-klotho group.

FIG. 7 shows immunostaining images of renal fibrosis indicators (α-SMA and vimentin) in mice in each group of the UIRI model. In FIG. 7, from left to right are the sham operation group, sham operation+mini-klotho group, UIRI group, and UIRI+mini-klotho group.

FIG. 8 shows expression levels of hepatic fibrosis-related proteins in mice in each group detected by immunoblotting in the CCL4 model. In FIG. 8, from left to right are the normal saline group, normal saline +mini-klotho group, CCL4 model group, and CCL4 model+mini-klotho group.

FIG. 9 shows Masson's trichrome staining images of mouse livers in each group of the CCL4 model. In FIG. 9, from left to right are the normal saline group, normal saline+mini-klotho group, CCL4 model group, and CCL4 model+mini-klotho group.

FIG. 10 shows expression levels of pulmonary fibrosis-related proteins in mice in each group detected by immunoblotting in the BLM model. In FIG. 10, from left to right are the sham operation group, sham operation+mini-klotho group, BLM group, and BLM+mini-klotho group.

FIG. 11 shows Masson's trichrome staining images of lungs of mice in each group of the BLM model. In FIG. 11, from left to right are the sham operation group, sham operation+mini-klotho group, BLM group, and BLM+mini-klotho group.

### DETAILED DESCRIPTION

The present disclosure will be further described below only by way of examples in conjunction with the drawings.

Construction of polypeptide mini-klotho: 159 amino acids of the KL1 domain of full-length Klotho are intercepted to obtain the polypeptide mini-klotho sequence. The amino acid sequence information of the polypeptide mini-klotho is as follows:

(SEQ ID NO. 1)
FQGTFPDGFLWAVGSAAYQTEGGWQQHGKGASIWDTFTHHPLAPPGDSRN
ASLPLGAPSPLQPATGDVASDSYNNVFRDTEALRELGVTHYRFSISWARV
LPNGSAGVPNREGLRYYRRLLERLRELGVQPVVTLYHWDLPQRLQDAYGG
WANRALADH.

In vitro cell experiments are conducted using a lentivirus overexpressing mini klotho with a Flag tag synthesized by Beijing Tsingke Biotech Co., Ltd.; In vivo animal experiments are conducted using an adeno-associated virus overexpressing mini-klotho with a GFP tag synthesized by Cyagen Biomodels (Guangzhou) Co., Ltd.

Example 1: Inhibitory Effect of Polypeptide Mini-Klotho on the Activation of Rat Renal Fibroblasts (NRK-49F) in Vitro

### 1. Experimental Materials

Cell: rat renal fibroblasts (NRK-49F).
Culture medium: DMEM/F12 (HAM) (1:1) culture medium containing 10% FBS.
Cultivation conditions: at 37° C. in an incubator containing 5% CO₂.

Construction of a small molecule polypeptide mini-klotho: 159 amino acids of the KLI domain of full-length Klotho were intercepted and submitted to Beijing Tsingke Biotech Co., Ltd. to synthesize a lentivirus with a Flag tag to overexpress mini-klotho.

### 2. Experimental Treatment

(1) The cultured rat renal fibroblasts (NRK-49F) were inoculated into a 6-well cell culture plate at a density of 1.5×10⁶ cells/well and cultured for 1 day, and transfected with a lentivirus at a multiplicity of infection (MIO) of 20 as an optimal level of infectious virus. The culture medium was changed after 24 h, and puromycin was screened for a stable cell line. The overexpressed mini-klotho lentiviral vector contained a puromycin resistance gene. In the preliminary experiment, all the untreated cells were killed in 3-4 days to obtain an optimal screening concentration of 7.5 μg/mL. The successfully transfected rat fibroblasts (NRK-49F) were screened with puromycin at a dose of 7.5 μg/mL until no cell death occurred, and a stable cell line overexpressing mini-klotho was established.
(2) The stable cell line and untreated rat renal fibroblasts (NRK-49F) were inoculated into a 6-well cell culture plate at a density of 1.5×10⁶ cells/well, and the basic medium was changed next day to starve the two groups of cells for 24 h. TGF-β (2 ng/ml), FGF2 (10 ng/ml), and Wnt3a (100 ng/ml) were added to stimulate respectively. After 24 hours, the proteins were collected and Western blot experiments were performed.

### 3. Experimental Results

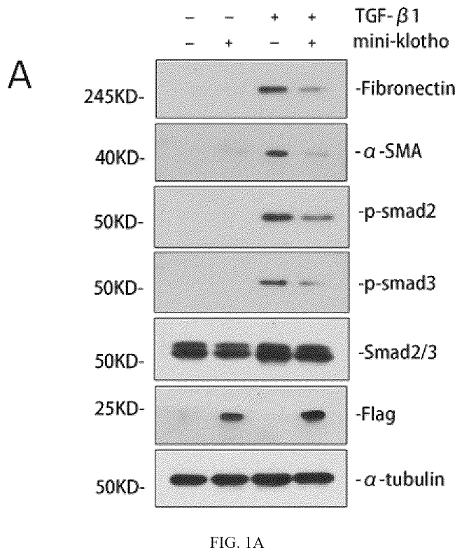

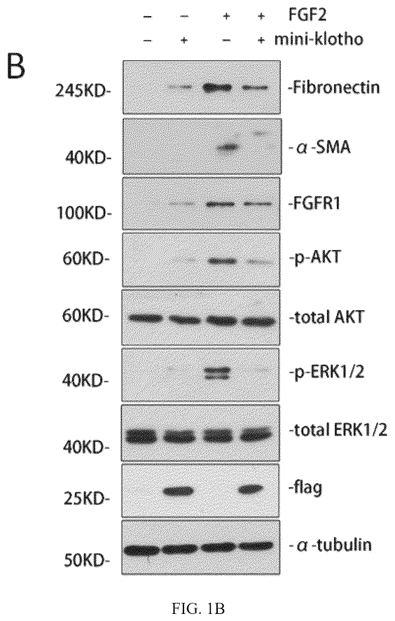

(1) The experimental results were shown in FIG. 1A. Mini-klotho could inhibit the expression of downstream molecules of the TGF-| pathway, as well as the expression of the mediated fibronectin and α-smooth muscle actin (α-SMA), and the expression of the Flag tag was detected to determine the successful overezpression of mini-klotho.
(2) As shown in FIG. 1B, mini-klotho can inhibit the expression of downstream molecules of the FGF2 pathway and the expression of the mediated fibrosis-related proteins.
(3) As shown in FIG. 1C, mini-klotho can inhibit the expression of downstream molecules of the Wnt3a pathway and the expression of the mediated fibrosis-related genes. Therefore, mini-klotho, as a novel polypeptide, can have antagonistic effects on TGF-β, FGF2 and Wnt3a, block key pro-fibrogenic signaling pathways in vivo, and thus inhibit the expression of fibrosis-related proteins.

### Example 2: Myo4A-AAV-Mini-Klotho-GFP was Specifically Expressed in the Muscles of UUO Mice with Intramuscular Injection

### 1. Experimental Animals

C57 mice, male, body weight 20-22 g, SPF grade.

First, the animals were weighed and numbered, and 24 healthy mice weighing 20-22 g were selected and randomly divided into 4 groups, with 6 mice in each group, including a sham operation group, a sham operation and mini-klotho injection group, a model group, and a model and mini-klotho injection group.

### 2. Experimental Grouping

(1) Sham operation group/sham operation and mini-klotho injection group: mice were anesthetized with 1% sodium pentobarbital at a dose of 1 mL/kg body weight at room temperature, and a 2-3 cm incision was made along the abdominal midline. After local disinfection, the skin, subcutaneous tissue, muscle layer and peritoneum were incised layer by layer. Once the left ureter was found, the incision was immediately sutured layer by layer. After local disinfection, the markings were verified, and the mice were placed in corresponding cages.
(2) Model group/model mini-klotho injection group: anesthesia and disinfection were performed as above. The skin, subcutaneous tissue, muscle layer and peritoneum were incised layer by layer. Once the left ureter was found, the ureter was ligated at the lower kidney and the upper ⅓ of the ureter, and the incision was immediately sutured layer by layer. After local disinfection, the markings were verified, and the mice were placed in the corresponding cages.

### 3. Experimental Process

The adeno-associated virus overexpressing mini-klotho was diluted with a sterile PBS solution to a titer of 10¹² gc/mL and was ready for use. Two weeks before surgery, 50 μl of adeno-associated virus overexpressing mini-klotho was injected into the right quadriceps muscle of the mice in the sham operation mini-klotho injection group and the model mini-klotho injection group at multiple points in situ. Mice in the sham operation group and model group were injected with 50 μl of empty virus vector. Each group of mice was bred in separate cages. UUO surgery was performed 2 weeks later, and the mice of each group were killed after 7 days of feeding. The left and right muscles, left and right kidneys, heart, liver, spleen, lungs and other organs were harvested and fixed in 10% neutral buffered formaldehyde and frozen in liquid nitrogen respectively. After the formaldehyde-fixed tissues were dehydrated, embedded, sliced, and sectioned, the GFP fluorescence intensity of left and right muscles, left and right kidneys, heart, liver, spleen, lungs and other organs were measured respectively.

### 4. Experimental Results

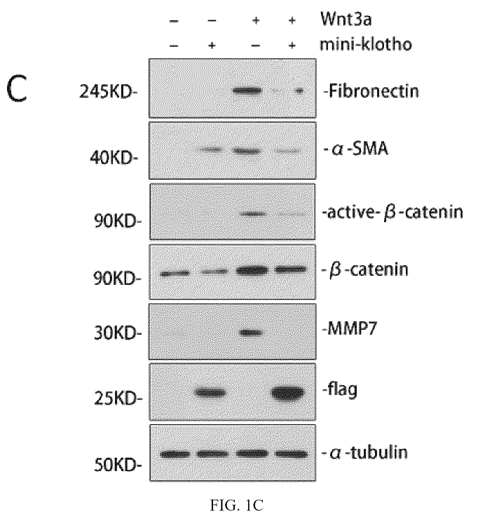

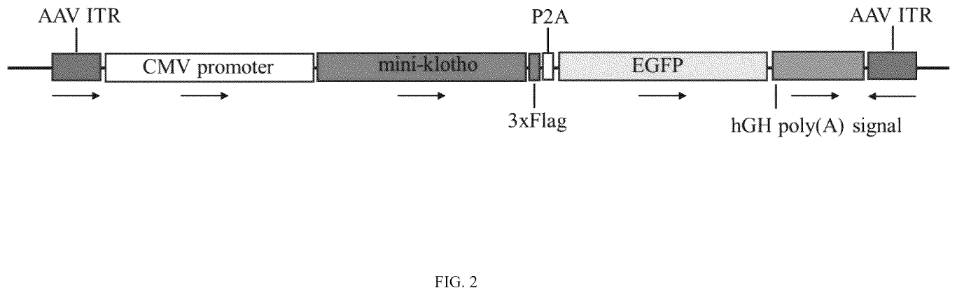

(1) FIG. 2 was a structural model diagram of Myo4A-AAV-mini-klotho-GFP virus.
(2) Mini-klotho-GFP was specifically expressed in the right muscle of mice:

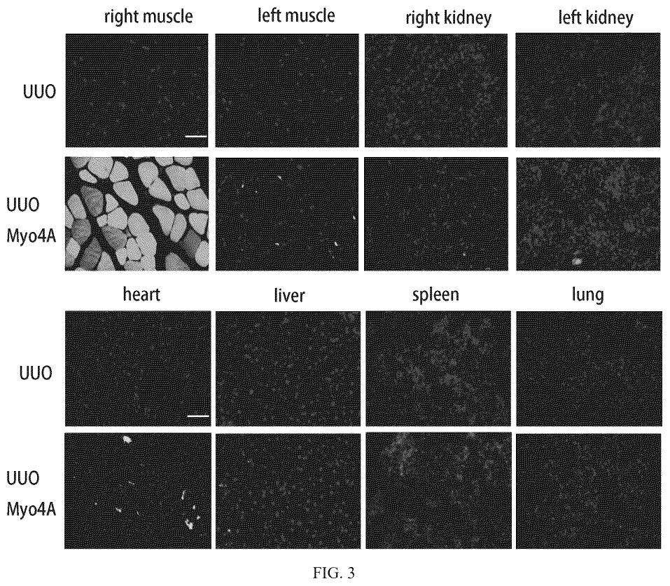

As shown in FIG. 3, mini-klotho-GFP was mainly expressed specifically in muscles, with a small amount of expression in the heart, liver, and kidneys. However, the organs of mice injected with empty virus vector showed no fluorescence, scale bar, 100 μm.

### Example 3: Inhibitory Effect of Polypeptide Mini-Klotho on Renal Fibrosis in Mouse UUO Model

### 1. Experimental Animals

C57 mice, male, body weight 20-22g, SPF grade.

First, the animals were weighed and numbered, and 24 healthy mice weighing 20-22 g were selected and randomly divided into 4 groups, with 6 mice in each group, including a sham operation group, a sham operation and mini-klotho injection group, a model group, and a model and mini-klotho injection group.

### 2. Experimental Grouping

(1) Sham operation group/sham operation and mini-klotho injection group: mice were anesthetized with 1% sodium pentobarbital at a dose of 1 mL/kg body weight at room temperature, and a 2-3 cm incision was made along the abdominal midline. After local disinfection, the skin, subcutaneous tissue, muscle layer and peritoneum were incised layer by layer. Once the left ureter was found, the incision was immediately sutured layer by layer. After local disinfection, the markings were verified, and the mice were placed in corresponding cages.
(2) Model group/model mini-klotho injection group: anesthesia and disinfection were performed as above. The skin, subcutaneous tissue, muscle layer and peritoneum were incised layer by layer. Once the left ureter was found, the ureter was ligated at the lower kidney and the upper ⅓ of the ureter, and the incision was immediately sutured layer by layer. After local disinfection, the markings were verified, and the mice were placed in the corresponding cages.

### 3. Experimental Process

The adeno-associated virus overexpressing mini-klotho was diluted with a sterile PBS solution to a titer of 10¹² gc/mL and was ready for use. Two weeks before surgery, 50 μl of adeno-associated virus overexpressing mini-klotho was injected into the right quadriceps muscle of the mice in the sham operation mini-klotho injection group and the model mini-klotho injection group at multiple points in situ. Mice in the sham operation group and model group were injected with 50 μl of empty virus vector. Each group of mice was bred in separate cages. UUO surgery was performed 2 weeks later, and the mice of each group were killed after 7 days of feeding. The left and right kidneys were harvested and fixed in 10% neutral buffered formaldehyde and frozen in liquid nitrogen respectively. After the formaldehyde-fixed tissues were dehydrated, embedded, sliced, and sectioned, they were stained with Masson and immunostained with fibronectin and α smooth muscle actin (α-SMA).

### 4. Experimental Results

(1) Masson staining showed that mini-klotho reduced renal interstitial collagen deposition in UUO mice:

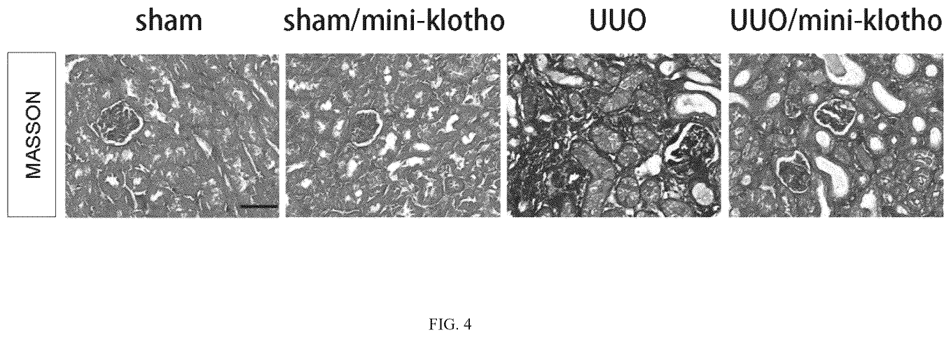

The experimental results were shown in FIG. 4. The renal interstitial collagen deposition of mice in the model mini-klotho injection group was significantly lower than that in the model group, scale bar, 50 μm.
(2) Mini-klotho reduced renal interstitial fibrosis in UUO mice:

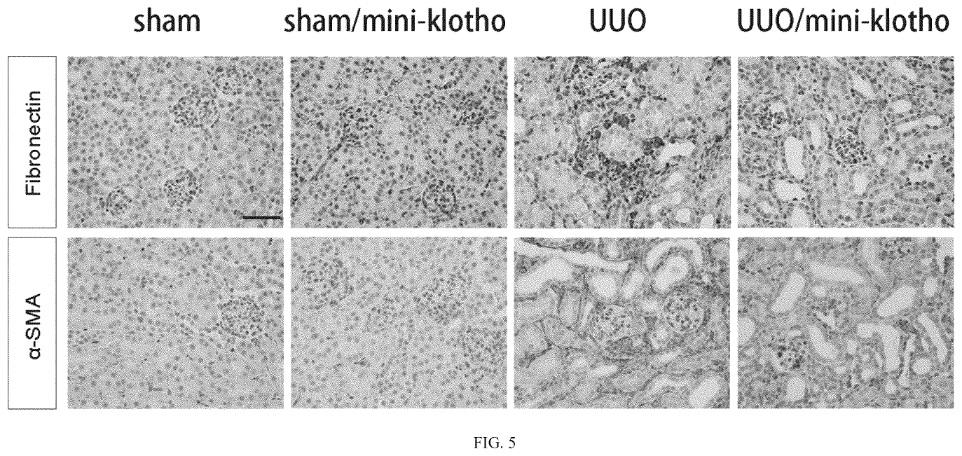

The experimental results were shown in FIG. 5. Compared with the model group, the levels of renal interstitial fibronectin and α-SMA in the model mini-klotho injection group were significantly reduced, scale bar, 50 μm.

### Example 4: Inhibitory Effect of Polypeptide Mini-Klotho on Renal Fibrosis in Mouse UIRI Model

### 1. Experimental Animals

C57 mice, male, body weight 20-22 g, SPF grade.

First, the animals were weighed and numbered, and 24 healthy mice weighing 20-22 g were selected and randomly divided into 4 groups, with 6 mice in each group, including a sham operation group, a sham operation and mini-klotho injection group, a model group, and a model and mini-klotho injection group.

### 2. Experimental Grouping

(1) Sham operation group/sham operation and mini-klotho injection group: mice were anesthetized with 1% sodium pentobarbital at a dose of 1 mL/kg body weight at room temperature, and a 2-3 cm incision was made along the abdominal midline. After local disinfection, the skin, subcutaneous tissue, muscle layer and peritoneum were incised layer by layer. Once the left renal pedicle was found, the incision was immediately sutured layer by layer. After local disinfection, the markings were verified, and the mice were placed in corresponding cages.
(2) Model group/model mini-klotho injection group: anesthesia and disinfection were performed as above. The skin, subcutaneous tissue, muscle layer and peritoneum were incised layer by layer. Once the left renal pedicle was found, it was quickly blocked with a non-damaging mini-artery clip. The kidney turned from bright red to purplish black, indicating successful clamping. The mice were placed on a metal heating plate at 37.5° C. for 35 min, and the incision was sutured layer by layer after the operation. After local disinfection, the markings were verified, and the mice were placed in corresponding cages.

### 3. Experimental Process

The adeno-associated virus overexpressing mini-klotho was diluted with a sterile PBS solution to a titer of 10¹² gc/mL and was ready for use. Two weeks before surgery, 50 μl of adeno-associated virus overexpressing mini-klotho was injected into the right quadriceps muscle of the mice in the sham operation mini-klotho injection group and the model mini-klotho injection group at multiple points in situ. Mice in the sham operation group and model group were injected with 50 μl of empty virus vector. UIRI surgery was performed 2 weeks later, and the mice of each group were killed after 10 days of feeding to collect blood samples. The left kidneys were all harvested and fixed in 10% neutral buffered formaldehyde and frozen in liquid nitrogen respectively. After the formaldehyde-fixed tissues were dehydrated, embedded, sliced, and sectioned, they were stained with Masson and immunostained with α-SMA and vimentin.

### 4. Experimental Results

(1) Masson staining showed that mini-klotho reduced renal interstitial collagen deposition in UIRI mice:

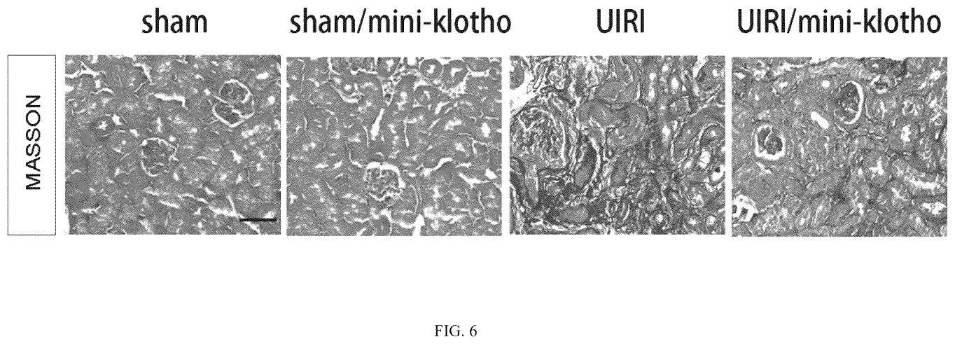

The experimental results were shown in FIG. 6. The renal interstitial collagen deposition of mice in the model mini-klotho injection group was significantly lower than that in the model control group, scale bar, 50 μm.
(2) Mini-klotho reduced renal interstitial fibrosis in UIRI mice:

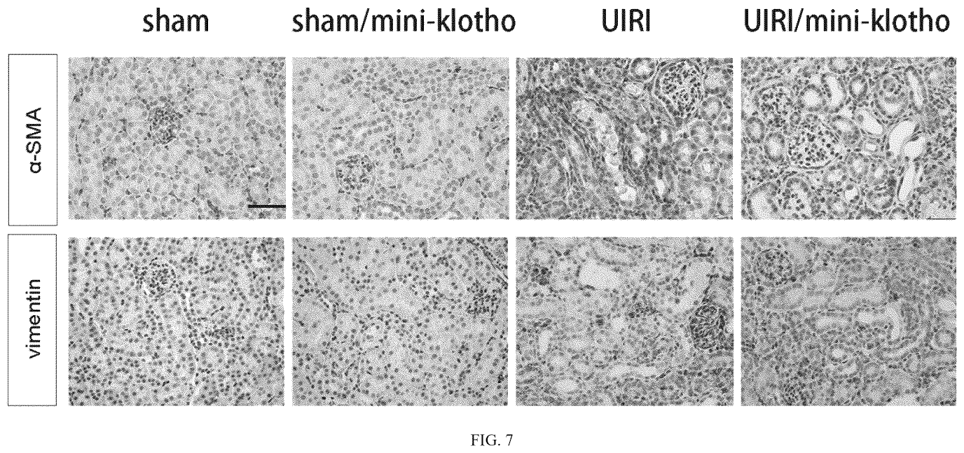

The experimental results were shown in FIG. 7. Compared with the model group, the levels of α-SMA and vimentin in the kidney tissue of mice in the model mini-klotho injection group were significantly reduced, scale bar, 50 μm.

### Example 5: Inhibitory Effect of Mini-Klotho on Hepatic Fibrosis in Mouse Carbon Tetrachloride (CCL4) Model

### 1. Experimental Animals

C57 mice, male, body weight 20-22 g, SPF grade.

First, the animals were weighed and numbered, and 24 healthy mice weighing 20-22 g were selected and randomly divided into 4 groups, with 6 mice in each group, including a normal saline group, a normal saline mini-klotho group injection, a CCL4 model group, and a CCL4 model mini-klotho injection group.

### 2. Experimental Grouping

(1) Normal saline group/normal saline mini-klotho group injection: each mouse was intraperitoneally injected with sterile physiological saline at a dose of 50 μl/20g every three days for 8 weeks. After injection, the markings were verified, and the mice were placed in the corresponding cages.
(2) CCL4 model group and CCL4 model mini-klotho group injection: the CCL4 solution was diluted with olive oil in a 1:3 volume ratio, and each mouse was intraperitoneally injected with the CCL4 solution at a dose of 50 μl/20 g every three days for 8 weeks. The markings were verified, and the mice were placed in the corresponding cages.

### 3. Experimental Process

The adeno-associated virus overexpressing mini-klotho was diluted with a sterile PBS solution to a titer of 10¹² gc/mL and was ready for use. Two weeks before CCL4 injection, 50 μl of adeno-associated virus overexpressing mini-klotho was injected into the right quadriceps muscle of the mice in the normal saline mini-klotho injection group and the CCL4 model mini-klotho injection group at multiple points in situ. Mice in the normal saline group and CCL4 model group were injected with 50 μl of empty virus vector. After 2 weeks, intraperitoneal injections of CCL4 and normal saline were performed every 3 days. At the 8th week, the mice of each group were killed to collect blood samples. The livers were all taken, and were fixed in 10% neutral buffered formaldehyde and frozen in liquid nitrogen. After the formaldehyde-fixed tissues were dehydrated, embedded, sliced, and sectioned, they were subjected to Masson staining and fibronectin immunostaining. The protein was extracted from frozen tissue homogenate, and the expression levels of fibrosis-related proteins were detected by Western Blot.

### 4. Experimental Results

(1) Mini-klotho reduced the expression levels of hepatic fibrosis-related proteins in CCL4 mice:

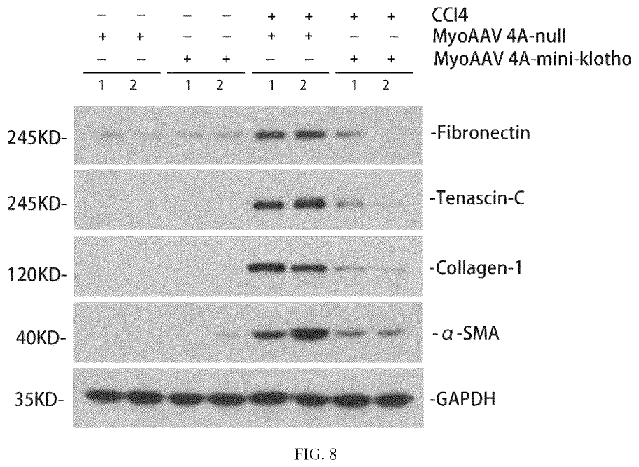

The experimental results were shown in FIG. 8. The expression levels of hepatic fibrosis-related proteins such as fibronectin, tenascin, collagen fiber I, vimentin, and α-SMA in mice in the CCL4 model mini-klotho injection group were significantly reduced.
(2) The degree of hepatic fibrosis was detected by Masson staining:

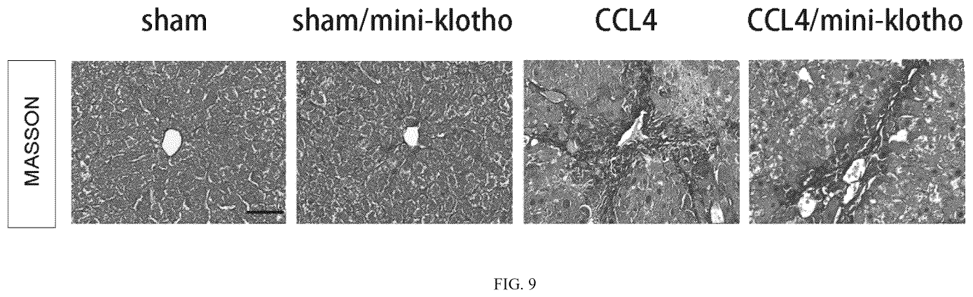

The experimental results were shown in FIG. 9. The collagen deposition in the liver of mice in the CCL4 model mini-klotho injection group was significantly lower than that in the CCL4 model group, scale bar, 50 μm.

### Example 6: Inhibitory Effect of Polypeptide Mini-Klotho on Pulmonary Fibrosis in Mouse Bleomycin (BLM) Model

### 1. Experimental Animals

C57 mice, male, body weight 20-22 g, SPF grade.

First, the animals were weighed and numbered, and 24 healthy mice weighing 20-22 g were selected and randomly divided into 4 groups, with 6 mice in each group, including a sham operation group, a sham operation mini-klotho injection group, a BLM model group and a BLM model mini-klotho injection group.

### 2. Experimental Groups

(1) Sham operation group/sham operation mini-klotho group injection: after the mice were anesthetized with 1% sodium pentobarbital at 1 mL/kg body weight at room temperature, a 1 cm incision of neck was selected. After local disinfection, the skin, subcutaneous tissue, and muscle layer were incised layer by layer. The connective tissue in front of the trachea was cleared to expose the trachea. The front end of a 1 mL insulin syringe was placed toward the heart, and inserted straight into the trachea through the middle of the two tracheal cartilage rings. 50 μl of normal saline was injected into the trachea. During this period, attention was paid to whether the nasal cavity of the mouse was choked with liquid. After the injection, the mouse was placed in an upright position with its head up, and the surgical board was struck on the table for 30 seconds. The mouse was removed from the surgical board, grabbed its upper limbs, and rotated for 2 min to allow the injected BLM to be evenly distributed in the lungs. The neck incision was then sutured layer by layer. After local disinfection, the markings were verified, and the mice were placed in the corresponding cages.
(2) BLM model group and BLM model mini-klotho injection group: anesthesia and disinfection were performed as above. After local disinfection, the skin, subcutaneous tissue, and muscle layer were incised layer by layer. The connective tissue was cleared in front of the trachea to expose the trachea. The front end of the 1 ml insulin syringe was placed toward the heart, and inserted straight into the trachea through the middle of the two tracheal cartilage rings. 50 μl of normal saline were injected into the trachea. During this period, attention was paid to whether the nasal cavity of the mouse was choked with liquid. After the injection, the mouse was placed in an upright position with its head up, and the surgical board was struck on the table for 30 seconds. The mouse was removed from the surgical board, grabbed its upper limbs, and rotated for 2 min to allow the injected BLM to be evenly distributed in the lungs. The neck incision was then sutured layer by layer. After local disinfection, the markings were verified, and the mice were placed in the corresponding cages.

### 3. Experimental Process

The adeno-associated virus overexpressing mini-klotho was diluted with the sterile PBS solution to a titer of 10¹² gc/ml and was ready for use. Bleomycin solution was diluted with sterile saline at a ratio of 1:50 at a concentration of 5 mg/kg. After injecting bleomycin solution and normal saline into the trachea, the virus was injected, where 50 μl of adeno-associated virus overexpressing mini-klotho were injected into the right quadriceps muscle of the mice in the sham operation injection group and the model injection group at multiple points in situ. Mice in the sham operation injection group and the model injection group were injected with 50 μl of empty virus vector. At the 4th week, the mice of each group were killed. The lungs were taken and fixed in 10% neutral buffered formaldehyde and frozen in liquid nitrogen. After the formaldehyde-fixed tissues were dehydrated, embedded, sliced, and sectioned, they were subjected to Masson staining and fibronectin immunostaining. The protein was extracted from frozen tissue homogenate, and the expression levels of fibrosis-related proteins were detected by Western Blot.

### 4. Experimental Results

(1) Mini-klotho reduced the expression levels of pulmonary fibrosis-related proteins in BLM mice:

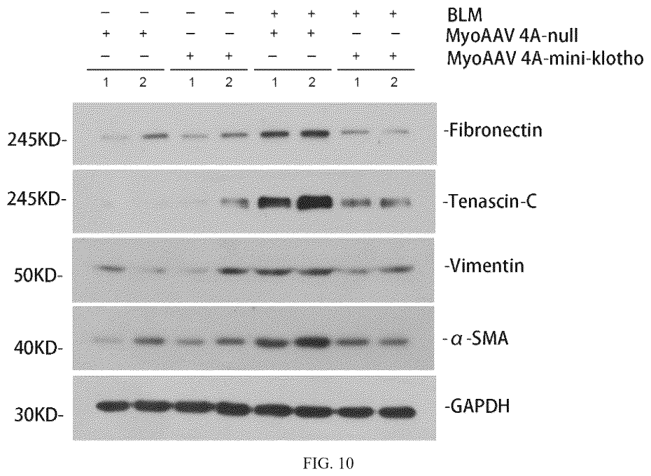

The experimental results were shown in FIG. 10. The expression levels of the pulmonary fibrosis-related proteins such as fibronectin, tenascin, vimentin, and α-SMA in mice in the model mini-klotho injection group were significantly reduced.
(2) The degree of pulmonary fibrosis was detected by Masson staining:

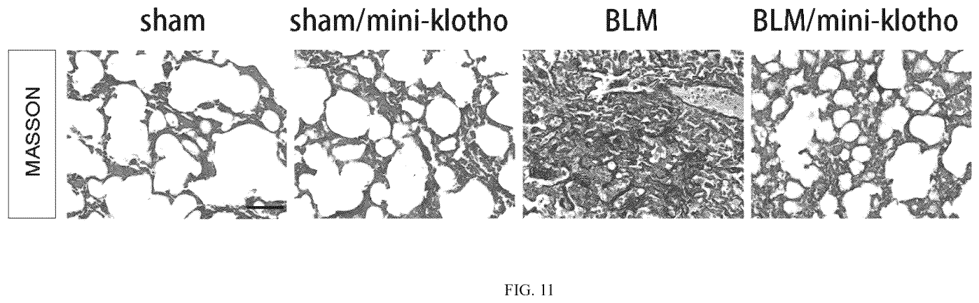

The experimental results were shown in FIG. 11. The collagen deposition in the lungs of mice in the model mini-klotho injection group was significantly lower than that in the model group, scale bar, 50 μm.

In summary, mini-klotho can significantly reduce collagen deposition in the kidney, liver, and lung tissues of UUO, UIRI, CCL4, and BLM mice, and significantly reduce expression levels of fibronectin, collagen I, and α-SMA in kidney, liver, and lung tissues of UUO, UIRI, CCL4, and BLM mice. Therefore, mini-klotho can become a new drug that effectively inhibits organ fibrosis.

1. A method for the treatment of organ fibrosis, comprising administering a therapeutically effective amount of a polypeptide mini-klotho or its derivatives to a subject in need thereof;
wherein amino acid sequences of the polypeptide mini-klotho are shown in SEQ ID NO. 1:
(SEQ ID NO. 1)
FQGTFPDGFLWAVGSAAYQTEGGWQQHGKGASIWDTFTHHPLAPPGDSRN
ASLPLGAPSPLQPATGDVASDSYNNVFRDTEALRELGVTHYRFSISWARV
LPNGSAGVPNREGLRYYRRLLERLRELGVQPVVTLYHWDLPQRLQDAYGG
WANRALADH.

2. The method according to claim 1, wherein the organ fibrosis is selected from the group consisting of renal fibrosis, hepatic fibrosis or pulmonary fibrosis.

3. The method according to claim 1, wherein the organ fibrosis is selected from renal interstitial fibrosis.

4. The method according to claim 1, wherein the organ fibrosis is selected from renal interstitial fibrosis caused by unilateral ureteral obstruction.

5. The method according to claim 1, wherein the organ fibrosis is selected from renal interstitial fibrosis caused by unilateral ischemia-reperfusion injury.

6. The method according to claim 1, wherein the organ fibrosis is selected from renal interstitial fibrosis caused by unilateral ureteral obstruction and unilateral ischemia-reperfusion injury.

7. The method according to claim 1, wherein the organ fibrosis is selected from hepatic fibrosis caused by carbon tetrachloride.

8. The method according to claim 1, wherein the organ fibrosis is selected from pulmonary fibrosis caused by bleomycin.

9. The method according to claim 1, wherein the polypeptide mini-klotho is administered by intramuscular injection.

10. The method according to claim 1, wherein derivatives of the polypeptide mini-klotho comprise shorter peptides containing amino acid sequences of mini-klotho, mini-klotho related polypeptides with amino acid substitutions, chemically modified mini-klotho and shorter peptides thereof.

11. A polypeptide mini-klotho or its derivatives, wherein amino acid sequences of the polypeptide mini-klotho are defined as in claim 1.

12. A drug comprising the polypeptide mini-klotho or its derivatives according to claim 11, and a myotropic Myo4A-AAV novel adeno-associated virus overexpressing the polypeptide mini-klotho.

13. A drug comprising a therapeutically effective dose of the polypeptide mini-klotho or its derivatives according to claim 11, and a myotropic Myo4A-AAV novel adeno-associated virus overexpressing the polypeptide mini-klotho.
